(19) United States
(12) Patent Application Publication
(10) Pub. No.: US 2001/0025134 A1
(43) Pub. Date: Sep. 27, 2001

(54) Catheter tip with bridged steering wire sleeve element

(76) Inventors: Edwin Bon, Canton, GA (US); Mahase Nardeo, Alpharetta, GA (US)

Correspondence Address: GARDNER GROFF MEHRMAN & JOSEPHIC, P.C., PAPER MILL VILLAGE, BUILDING 23, 600 VILLAGE TRACE, SUITE 300, MARIETTA, GA, 30067, US

(21) Appl. No.: 09/809,334

(22) Filed: Mar. 15, 2001

Related U.S. Application Data
(60) Provisional application No. 60/189,416, filed on Mar. 15, 2000.

(51) Int. Cl. A61B 1/01

(52) U.S. Cl. 600/146

(57) ABSTRACT

A steerable catheter having a tip assembly including bridged steering wire sleeves. A bridged steering wire sleeve element is provided for the tip assembly. The bridged steering wire sleeve element has a first sleeve engaged within a first steering wire lumen of the catheter shaft and having a first passage extending therethrough for passing a first steering wire portion, a second sleeve engaged within a second steering wire lumen of the catheter shaft and having a second passage extending therethrough for passing a second steering wire portion, and a bridge portion extending between said first and second sleeves.

### CROSS-REFERENCE TO RELATED APPLICATION

This application claims the benefit of U.S. provisional patent application Ser. No. 60/189,416, filed Mar. 15, 2000, which is incorporated herein by reference.

### BACKGROUND OF THE INVENTION

1. Field of the Invention

The present invention relates generally to medical devices, and more particularly to a steerable catheter having a tip assembly with a bridged steering wire sleeve component.

2. Description of Related Art

Medical practitioners frequently gain access to internal regions of a patient's body through the use of medical catheters in a variety of medical procedures, in order to reduce or eliminate the need for more invasive procedures. Medical catheters may be used to access internal body regions with a fiberoptic scope, light bundles, and/or other surgical instruments or devices, for a variety of diagnosis, treatment and/or material delivery purposes. For example, U.S. Pat. No. 5,658,263 to Dang, et al. discloses a multi-segmented guiding catheter typically utilized for internal vascular access. Steerable catheters have been developed to provide improved access to internal tissue. These catheters typically include a flexible catheter shaft and steering wires for controlling the flexure of the catheter shaft. Examples of steerable catheters are shown by U.S. Pat. Nos. 5,342,299 and 5,437,636 to Snoke, et al., and U.S. Pat. No. 5,199,950 to Schmitt, et al. U.S. Pat. No. 5,454,794 to Narcisco, et al. shows a steerable light diffusing catheter for treating luminal surfaces with photodynamic therapy. A mechanism for steering a catheter is disclosed by U.S. Pat. No. 5,456,664 to Heinzelman, et al. U.S. Pat. No. 5,396,880 to Kagan, et al. discloses an endoscope for accessing the spinal epidural space.

A problem common to many previously existing steerable catheters is the provision of acceptable flexibility of the shaft, particularly near the shaft tip, while still providing secure retention of the steering wire or wires within the catheter shaft. The steering wire or wires typically comprise a small diameter length of high tensile strength material, whereas the flexible catheter shaft material typically must be relatively soft in order to provide sufficient flexibility. Thus, in many instances, the steering wire will cut through the flexible catheter shaft material, or will otherwise become disattached from the catheter shaft, rendering the catheter inoperable or compromising its utility. Softer materials are desirable for use in construction of catheter shafts, due to their generally greater flexibility, but are more susceptible to steering wire pull-through or damage by the steering wire than are harder materials of construction. The problem of steering wire pull-through or damage by the steering wire is especially troublesome with small diameter catheters having one or more instrument access lumens therethrough, due to the reduced material thickness of the catheter. Previous attempts to provide acceptable flexibility of the shaft, particularly near the shaft tip, while maintaining secure retention of the steering wire or wires within the catheter shaft have met with varying degrees of success, but none have proven fully successful.

It has been discovered that the provision of wear-resistant sleeves surrounding the steering wires in the tip area of a catheter helps resist steering wire pull-through. For example, as disclosed in commonly assigned United States Patent application Ser. No. 09/211,345, filed Dec. 15, 1998, which is hereby incorporated herein by reference in its entirety, a pair of sleeves are provided in the tip surrounding each steering wire. By coining a segment of the steering wire along the loop of wire that extends between the two sleeves adjacent the tip of the catheter shaft, the cross-sectional dimension of the steering wire in the plane normal to the catheter shaft is reduced, while the cross-sectional dimension of the steering wire in a plane along the catheter shaft is increased. By reducing the cross-sectional dimension of the steering wire in the plane normal to the catheter shaft, the size of the instrument lumens may be maximized without obstruction by the loop of steering wire passing between the instrument lumens. By increasing the cross-sectional dimension of the steering wire in a plane along the catheter shaft, a positive mechanical stop is provided, preventing the looped segment of steering wire from freely passing through the sleeves, thereby further resisting steering wire pull-through and improving steering performance. The present invention further improves upon this aspect of a steerable catheter. Additional problems arise from the use of standard Touhy-Borst assemblies 10 as shown in FIG. 1, or other catheter manifolds such as that disclosed by U.S. Pat. No. 5,507,732 to McClure, et al., as catheter inlets. The standard Touhy-Borst assembly 10 typically comprises a two-piece housing, having a first housing component 12 coupled to a second housing component 14 by means of a threaded luer 16 or other coupling. The use of this type of two-piece housing results in increased assembly time and expense, and presents a risk of detachment during use. Additionally, the coupling 16 permits relative rotational movement between the first housing component 12 and the second housing component 14. This is disadvantageous as it is has been found desirable to maintain the flush port 18 in a fixed position relative to the catheter housing. The standard Touhy-Borst assembly 10 is typically affixed to the catheter housing by means of mounting wings 20 provided on the second housing component 14. Even if the wings 20 are rigidly attached to second housing component 14, the rotational movement permitted at coupling 16 allows movement of the first housing 1 component 12 and the flush port 18 thereof. In addition, the standard Touhy-Borst assembly 10 presents several steps or discontinuities 22a, 22b, 22c within its internal passage. These discontinuities present obstructions to instrument passage, and can result in abrasive wear and tear on sensitive instruments. The standard Touhy-Borst assembly 10 is also less than fully satisfactory for use as a catheter inlet housing, as it typically includes only two mounting wings 20. It has been found desirable to provide additional mounting wings angularly spaced about the circumference of the inlet housing for more accurate positioning. It has also been found desirable to increase the thickness and contact area of the mounting wings to provide more secure attachment to the catheter body housing than is permitted using a standard Touhy-Borst assembly 10. Other known manifold assemblies suffer disadvantages similar to those of the Touhy-Borst assembly.

Thus, it has been found that a need exists for an improved steerable catheter device. It is to this and other needs that the present invention is primarily directed.

### SUMMARY OF THE INVENTION

The steerable catheter was developed to satisfy a need that pain practitioners have faced for many years: the need to directly visualize pathology in-situ in and around the nerve roots as they make their way out of neural foramina along the axis of the spinal cord. Other means of visualizations such as fluoroscopy, MRI and CAT scans cannot provide real time images of pathology or disease and cannot clearly differentiate soft tissue pathology. Epidural endoscopy is difficult or impossible if performed utilizing rigid optics and the paramedian or lumbar approach. This is due to the fact that the spinal cord is encased by an articulating bone structure presenting minimal access possibilities. Any device with the capability to access the epidural space must be flexible and small. Using miniature fiberoptic endoscopes and miniature multi lumen steerable catheters the present invention provides a medical device that may be introduced into the epidural space. The present invention allows physicians to directly visualize the epidural space of the spine and treat patients for related disease in a minimally invasive manner.

The present invention provides a steerable video guided catheter having lumens for instrument access, and providing the ability to steer the catheter tip in one or more planes. The present invention optionally also can provide a kit including disposable products that are used by the physician to access the epidural space through the sacral hiatus (tail bone). The kit contains drapes, syringe, needles, introducer set. etc. The present invention optionally can also provide a video system including a CCD camera, light source, and video monitor. The CCD camera is used to pick up the optical image from the endocoupler and convert it to an electronic signal that is sent to the video monitor. The light source consists of a bright light that is focused on the light fiber bundle to transmit light to the distal end of the scope.

In a preferred form, the present invention provides a tip assembly for a steerable catheter, which assembly includes a catheter shaft having first and second steering wire lumens extending lengthwise therethrough. One or more access lumens may optionally be provided through the length of the catheter shaft, for permitting passage of a fiberoptic endoscope and other instruments, infused fluids, aspirated materials, and/or otherwise accessing internal regions. The tip assembly preferably further includes a continuous length of steering wire having a first leg extending through the first steering wire lumen, a second leg extending through the second steering wire lumen, and a generally U-shaped looped segment connecting the first and second legs. The looped segment of steering wire may optionally include a coined or otherwise formed expanded outer dimension. A first wear-resistant sleeve is preferably provided within the first steering wire lumen adjacent the looped segment of steering wire, and a second wear-resistant sleeve is preferably provided within the second steering wire lumen adjacent the looped segment of steering wire. The wear-resistant sleeves and coined portion of the steering wire cooperate to provide improved resistance against steering wire pull-through or detachment from the material of the catheter shaft. In further preferred embodiments, the wear-resistant sleeves and coined portion function in cooperation with a segmented catheter shaft to provide improved flexibility without sacrificing resistance to steering wire pull-through.

In another aspect, the present invention preferably comprises a segmented catheter shaft for a steerable catheter. The segmented catheter shaft preferably includes a first shaft segment having a rear distal end for connection to a catheter body housing, and a front distal end opposite the rear distal end. The segmented catheter shaft preferably further includes a tip segment having a first end fused to the front distal end of the first shaft segment, and a second end opposite the first end. The first shaft segment is preferably relatively stiff to prevent buckling of the catheter shaft, whereas the tip segment is relatively flexible, as compared to the first shaft segment, for improved steerability. The segmented catheter shaft preferably further includes an end segment having a first end fused to the second end of the tip segment, and a second end opposite the first end. In preferred form, the segmented catheter shaft also includes first and second steering wire lumens extending lengthwise through the first shaft segment, the tip segment, and the end segment; and optionally includes one or more access lumens extending throughout the length of the catheter shaft. A continuous length of steering wire is preferably provided, having a first leg extending through the first steering wire lumen and a second leg extending through the second steering wire lumen. A looped segment of the length of steering wire preferably connects the first and second legs, extending across the second end of the end segment of the catheter shaft. A cover segment is preferably also provided, overlying at least a portion of the second end of the end segment, and encapsulating the looped segment of steering wire between the second end of the end segment and the cover segment. The end segment and cover segment are preferably formed of relatively stiff materials of construction, as compared to the tip segment, to resist steering wire pull-through. Wear-resistant sleeves can be provided in the steering wire lumens of the end segment to provide improved resistance to steering wire pull-through.

The present invention further comprises a number of alternative catheter tip assemblies, in addition to the presently preferred sleeved, continuous looped steering wire configuration. Alternative catheter tip assemblies of the present invention include: sleeveless, continuous looped steering wire configurations, optionally providing the looped portion of the steering wire with a coined or otherwise expanded dimension; multiple hooked steering wire configurations; multiple ballized steering wire configurations; multiple steering wire configurations incorporating wire jackets; multiple steering wire configurations incorporating wire washers; multiple steering wire configurations incorporating wire eyelets; multiple steering wire configurations incorporating wire springs; and bonded multiple steering wire configurations. Preferred embodiments of such alternative catheter tip assemblies are described in greater detail below.

Another aspect of the present invention provides a method of forming a segmented catheter shaft. The method preferably comprises providing a first shaft segment, a tip segment, and an end segment, each having first and second steering wire lumens extending lengthwise therethrough. One or more access lumens can optionally also be provided through the first shaft segment, the tip segment, and the end segment. Mandrels are inserted through the steering wire lumens and, if provided, the access lumens. The tip segment is arranged on the mandrels between the first shaft segment and the end segment. The first shaft segment, tip segment and end segment are then bonded end-to-end, to form a shaft assembly having a connecting end comprising a portion of the first shaft segment and a free end comprising a portion of the end segment. The mandrels are removed, and one or more steering wires are inserted through the shaft assembly. The steering wire is preferably a single, unitary, continuous length of wire having a first leg extending through the first steering wire lumen, a second leg extending through the second steering wire lumen, and a looped segment connecting the first and second legs adjacent the tip end of the shaft assembly. Less preferably, any of a variety of alternative catheter tip assemblies can be installed in the steering wire lumens. The provision of a continuous looped steering wire is preferable, as it provides improved resistance to steering wire pull-through in cooperation with the materials of the segmented catheter shaft of the present invention and, if provided, the wear-resistant sleeved and coined steering wire configuration of the present invention. The looped segment of steering wire is preferably encapsulated between the free end of the shaft assembly and a cover segment applied to overlie at least a portion of the free end of the shaft assembly.

In a further preferred embodiment, the looped segment of the steering wire is provided with an expanded outer dimension larger than an inner dimension of the first and second steering wire lumens, and/or wear-resistant sleeves can be installed in the steering wire lumens of the end segment prior to installing the steering wire. The first shaft segment and the end segment are preferably formed from a material having a first stiffness, and the tip segment from a material having a second stiffness less than the first stiffness. In a further preferred embodiment, a manifold is formed at the connecting end of the shaft assembly by inserting core pins into the steering wire lumens and, if provided, into the access lumen(s), and injection molding the manifold around the core pins. The core pins are then removed from the lumens upon demolding of the manifold. The provision of a manifold provides a degree of strain relief, and facilitates mounting of the shaft to the catheter body.

In a further improved embodiment, the present invention is a catheter tip assembly comprising at least one steering wire; and a bridged steering wire sleeve element including a first sleeve having a first passage extending therethrough for receiving a first steering wire portion, a second sleeve having a second passage extending therethrough for receiving a second steering wire portion, and a bridge portion extending between the first and second sleeves.

In another aspect, the present invention is a catheter comprising a flexible shaft with first and second steering wire lumens; at least one steering wire extending through the first and second steering wire lumens; and a bridged steering wire sleeve element comprising a first sleeve engaged within the first steering wire lumen and having a first passage extending therethrough for receiving a first steering wire portion, a second sleeve engaged within the second steering wire lumen and having a second passage extending therethrough for receiving a second steering wire portion, and a bridge portion extending between the first and second sleeves.

In another aspect, the present invention is a bridged steering wire sleeve element comprising a first sleeve having a first passage extending therethrough for receiving a first steering wire portion, a second sleeve having a second passage extending therethrough for receiving a second steering wire portion, and a bridge portion extending between the first and second sleeves.

In another aspect, the present invention is a method of resisting steering wire pull-through in a shaft of a steerable catheter, the method comprising providing a first sleeve engaged within a first steering wire lumen of the shaft and having a first passage extending therethrough receiving a first steering wire portion, providing a second sleeve 11 engaged within a second steering wire lumen of the shaft and having a second passage extending therethrough receiving a second steering wire portion, and providing a bridge portion extending between said first and second sleeves.

In another aspect, the present invention is a method of fabricating a catheter shaft, the method comprising providing a flexible shaft comprising first and second steering wire lumens; installing a bridged steering wire sleeve element comprising a first sleeve engaged within the first steering wire lumen and having a first passage extending therethrough, a second sleeve engaged within the second steering wire lumen and having a second passage extending therethrough, and a bridge portion extending between the first and second sleeves; and installing at least one steering wire into the first and second steering wire lumens and through the first and second passages of the bridged steering wire sleeve element.

Another aspect of the present invention provides an inlet housing for the catheter. The inlet housing preferably includes a unitary body portion having an instrument inlet, an outlet, an internal passage extending between the instrument inlet and the outlet, and an outer mounting surface. A sealing element is preferably provided adjacent the instrument inlet. In a further preferred embodiment, the inlet housing includes a flush port in fluid communication with the internal passage. One or more mounting flanges can be provided, extending outwardly from the unitary body portion. In a preferred embodiment, four mounting flanges are provided, spaced circumferentially about the unitary body portion at 90° intervals, with one of the mounting flanges generally aligned with the flush port. The internal passage of the inlet housing preferably provides a smooth transition between the instrument inlet and the outlet, whereby tools, instruments or other materials inserted therethrough will not meet with substantial obstruction.

A further aspect of the present invention provides a steerable catheter incorporating one or more of the above-described features. In a preferred form, the steerable catheter includes a catheter body having a steering actuator for steering an attached catheter shaft. The steerable catheter shaft preferably is formed from segments of different stiffnesses as described above, and includes first and second steering wire lumens and at least one access lumen extending lengthwise therethrough. The steerable catheter preferably includes a tip assembly comprising a continuous length of steering wire having a first leg extending through the first steering wire lumen, a second leg extending through the second steering wire lumen, and a looped segment connecting the first and second legs of the steering wire. The ends of the first and second legs opposite the looped segment are coupled to the steering actuator. Wear-resistant sleeves are preferably provided within the first and second steering wire lumens adjacent the looped segment of the steering wire. One or more inlet housings are preferably mounted to the catheter body housing, one inlet housing corresponding to each of the access lumens provided through the segmented catheter shaft. One or more access conduits are preferably also provided, coupling each inlet housing to its corresponding access lumen.

The entire scope and content of U.S. patent application Ser. No. 09/211,345, filed Dec. 15,1998; U.S. patent application Ser. No. 09/126,863, filed Jul. 31, 1998; and U.S. patent application Ser. No. 08/777,548, filed Dec. 30,1996, and now U.S. Pat. No. 6,030,360, issued Feb. 29, 2000, are hereby incorporated herein by reference.

These and other features and advantages of preferred forms of the present invention are described herein with reference to the drawing figures.

### BRIEF DESCRIPTION OF THE DRAWING FIGURES

FIG. 1 shows a side view, in partial cross-section of a prior art Touhy-Borst assembly.

FIG. 2 shows a top view of a steerable catheter according to a preferred form of the present invention.

FIG. 3 shows a side view of the steerable catheter shown in FIG. 2.

FIG. 4 shows a top view of a portion of the steerable catheter shown in FIG. 2, with the top cover removed to show internal components.

FIG. 5 shows a detailed view of certain internal components of the steerable catheter shown in FIG. 2, with the housing removed for clarity and shown generally in phantom lines.

FIG. 6 shows a plan view of a tip assembly of a steerable catheter, in partial cut-away view, according to one form of the present invention.

FIG. 7 shows a cross-sectional view, taken at line 7-7 of FIG. 6, of the tip assembly shown in FIG. 6.

FIG. 8 shows a plan view of a steering wire portion of the tip assembly shown in FIG. 6.

FIG. 9 shows an end view, from line 9-9 in FIG. 8, of the steering wire.

FIG. 10 shows a plan view of a tip assembly of a steerable catheter, in partial cut-away view, according to another form of the present invention.

FIG. 11 shows a cross-sectional view, taken at line 11-11 of FIG. 10, of the tip assembly shown in FIG. 10.

FIG. 12 shows a plan view of a steering wire portion of the tip assembly shown in FIG. 10.

FIG. 13 shows a side view, in partial cross-section, of an inlet housing according to a preferred form of the present invention.

FIGS. 14A-14D show an alternative catheter tip assembly having an unsleeved looped steering wire configuration with an expanded looped portion.

FIGS. 15A-15C show an alternative catheter tip assembly having an unsleeved looped steering wire configuration without an expanded looped portion.

FIGS. 16A-16C show an alternative catheter tip assembly having a multiple hooked steering wire configuration.

FIGS. 17A-17C show an alternative catheter tip assembly having a multiple ballized steering wire configuration.

FIGS. 18A-18C show an alternative catheter tip assembly having a multiple steering wire configuration incorporating wire jackets.

FIGS. 19A-19C show an alternative catheter tip assembly having a multiple steering wire configuration incorporating wire washers.

FIGS. 20A-20C show an alternative catheter tip assembly having a multiple steering wire configuration incorporating wire eyelets.

FIGS. 21A-21C show an alternative catheter tip assembly having a multiple steering wire configuration incorporating wire springs.

FIGS. 22A-22C show an alternative catheter tip assembly having a bonded multiple steering wire configuration.

FIG. 23 shows a perspective view of a catheter tip assembly including a bridged steering wire sleeve element according to a preferred form of the present invention.

FIG. 24 shows a perspective view of a catheter tip assembly including a bridged steering wire sleeve element according to another preferred form of the present invention. FIG. 25a shows a perspective view of a catheter tip assembly including a bridged steering wire sleeve element according to a preferred form of the present invention.

FIG. 25b shows a perspective view of a bridged steering wire element, according to a preferred form of the present invention.

FIG. 25c shows an end view of a bridged steering wire element, according to a preferred form of the present invention.

FIG. 26 shows a cross-sectional view, on a plane perpendicular to the catheter shaft, of a catheter tip assembly including a bridged steering wire sleeve element according to a preferred form of the present invention.

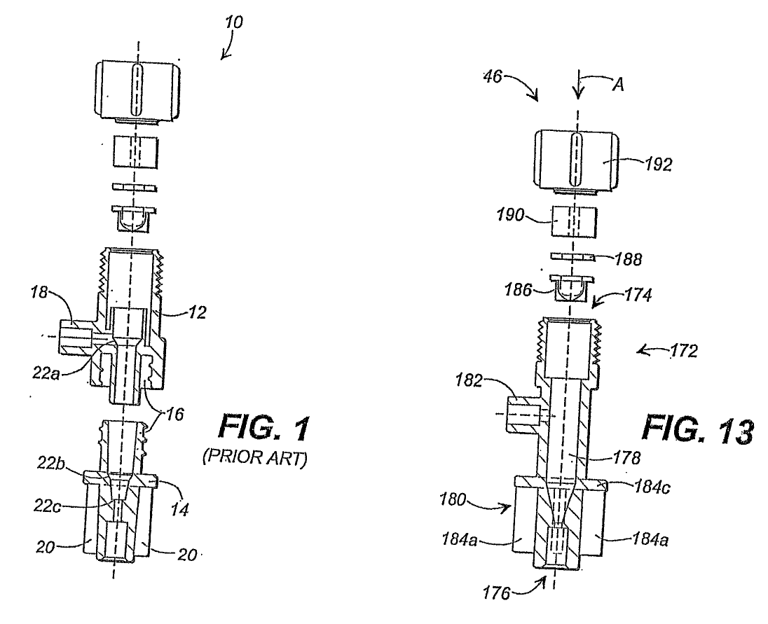

### DETAILED DESCRIPTION

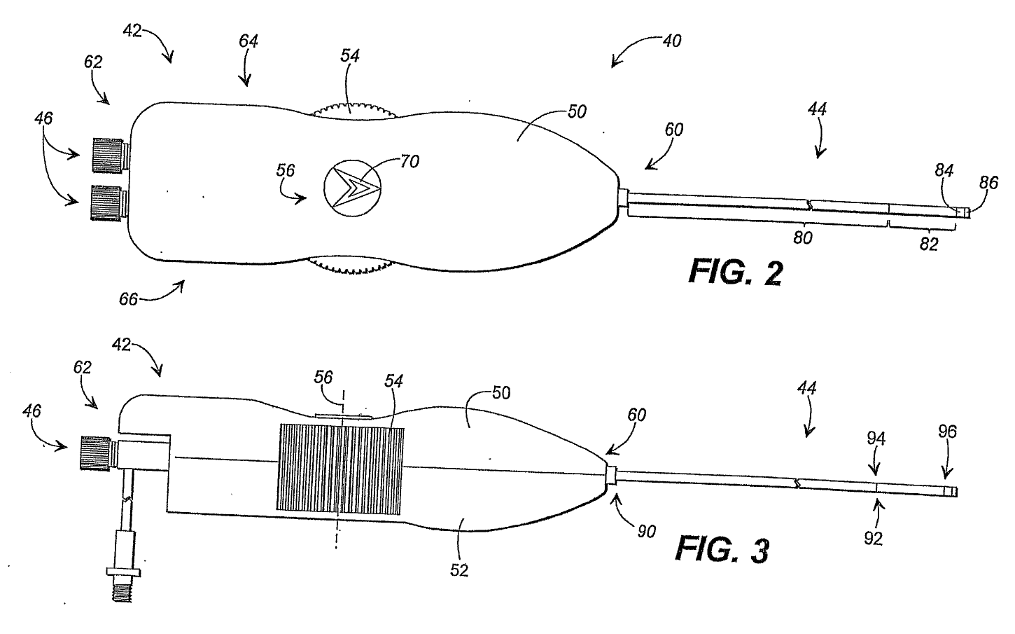

Referring now to the drawing figures, wherein like reference numerals represent like parts throughout, preferred forms of the present invention will now be described. As seen best with reference to FIGS. 2-5, the present invention generally comprises a steerable catheter 40, having a catheter body 42, a segmented catheter shaft 44, and at least one inlet housing 46. In a preferred form, the catheter body 42 comprises an upper housing shell 50 and a lower housing shell 52. A steering actuator such as, for example, a rotatably mounted dial 54 is preferably mounted in or on the catheter body 42. In the depicted embodiment, the dial 54 is rotatable about an axis 56 formed by cooperating projections and recesses on and in the dial 54 and one or both of the housing shells 50, 52. The dial 54 is preferably retained in place between the housing shells 50, 52, which are attached to one another in their assembled configuration by adhesive, thermal welding, and/or one or more couplings 58, such as cooperating crash pins having interengaging male and female portions, resilient couplings, screws, rivets or other fasteners. The catheter body 42 generally comprises a forward end 60, a rear end 62, and first and second sides 64, 66, and is generally sized and shaped to be comfortably manipulated by a practitioner's hand. The catheter body is preferably fabricated, as by injection molding, from plastic or another substantially rigid material of construction. One or more cutout sections 68 can be provided in one or both of the housing shells 50, 52 to provide clearance for the steering actuator. The housing shells 50, 52 can also be provided with engagement features and openings, as required for mounting of the inlet housings 46 and the catheter shaft 44, as is more fully discussed below. The steering actuator can additionally comprise directional indicating means 70 for visually indicating the deflection of the steering actuator, and thereby the expected degree of displacement of the steerable tip of the catheter shaft 44.

The segmented catheter shaft 44 of the present invention will be described with particular reference to FIGS. 2-10. The segmented catheter shaft 44 is preferably formed from PEBAX (polyether block amides) extrusions, or other flexible, biocompatible plastic materials. In a preferred embodiment depicted in the figures, the catheter shaft 44 generally comprises a first shaft segment 80, a tip segment 82, an end segment 84, and a cover segment 86. The first shaft segment 80 comprises a rear distal end 90 for connection to the forward end 60 of the catheter body 42, and a front distal end 92 generally opposite said rear distal end 90. The tip segment 82 preferably comprises a first end 94 fused or otherwise attached to the front distal end 92 of the first shaft segment 80, and a second end 96 generally opposite the first end 94. The end segment 84 preferably comprises a first end 98 fused or otherwise attached to the second end 96 of the tip segment 82 and a second end 100 generally opposite the first end 98. The cover segment 86 preferably overlies at least a portion of the second end 100 of the end segment 84.

In preferred form, the first shaft segment 80 is formed from a material having a first stiffness and the tip segment 82 is formed from a material having a second stiffness less than the first stiffness. In this manner, the first shaft segment 80 resists buckling along its length, and the relatively softer, more flexible tip segment 82 permits improved steerability. In a presently preferred example embodiment, the first shaft segment 80 is formed from a 7233 durometer PEBAX extrusion having a diameter of approximately 0.118 inch, and the tip segment 82 is formed from a 4033 durometer PEBAX extrusion of substantially identical cross-section and diameter. The end segment 84 is preferably formed from a material having a third stiffness greater than the second stiffness, for example, a 7233 durometer PEBAX extrusion substantially identical to that of the first shaft segment 80. The provision of the end segment 84 having substantially greater hardness than the tip segment 82 provides increased resistance to steering wire detachment during operation. The cover segment 86 is preferably formed from a relatively hard material such as 7233 durometer PEBAX, but can alternatively be formed from a softer material such as 4033 durometer PEBAX. The lengths of the segments of the catheter shaft 44 will vary depending upon the intended application. In a presently preferred example embodiment, the first shaft segment 80 is between 10″ to 14″, and most preferably approximately 11″ in length; the tip segment 82 is approximately 1.5″ in length; the end segment 84 is approximately 0.200″ to 0.500″ in length; and the cover segment 86 is approximately 0.200″ or less in length. The segments of the catheter shaft 44 are thermal welded or otherwise fused to one another end-to-end, with any internal lumens aligned between the segments, to form a shaft assembly 104 having a connecting end 106 comprising the rear distal end 90 of the first shaft segment 80, and a free end 108 comprising the end segment 84 and cover segment 86.

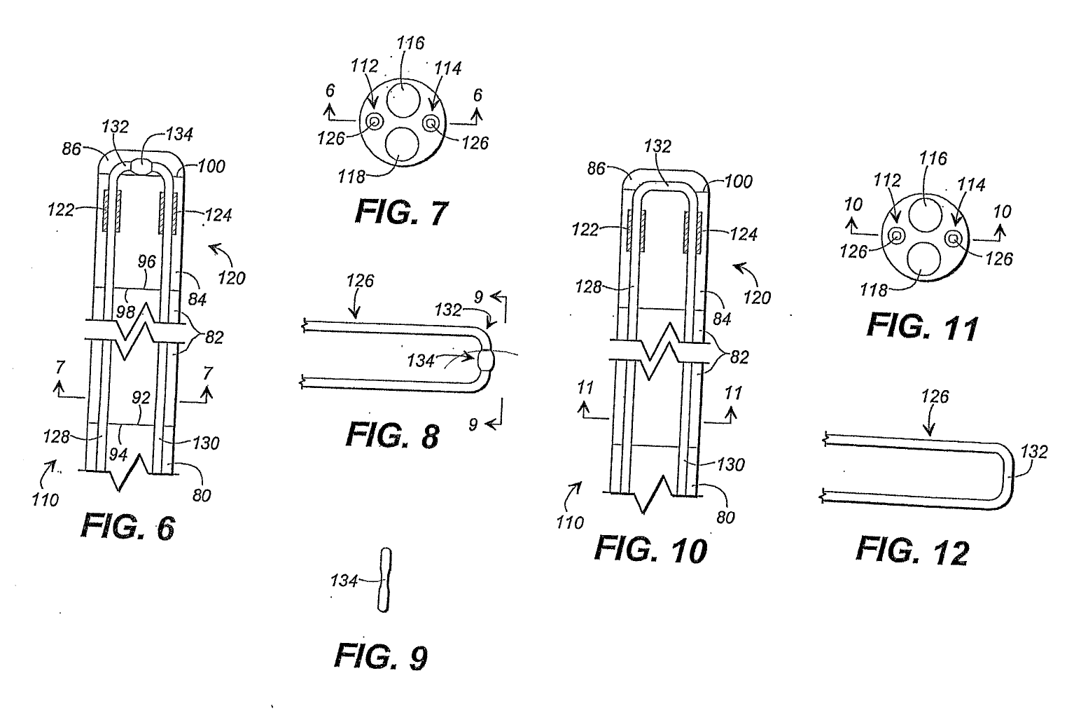

As seen best with reference to FIGS. 6-12, the catheter shaft 44 preferably comprises first and second steering wire lumens 112, 114 extending lengthwise through the first shaft segment 80, the tip segment 82, and the end segment 84. The diameter of the steering wire lumens 112, 114 may vary depending upon the intended application. In a presently preferred embodiment, the diameter of the steering wire lumens 112, 114 is approximately 0.014 inch. The catheter shaft 44 preferably has a generally round cross-section, as seen best with reference to FIGS. 7 and 11. The steering wire lumens 112, 114 are arranged generally diametrically opposite one another along a first diameter of the cross-section. In a further preferred embodiment, the catheter shaft 44 optionally further comprises one or more (two are shown) access lumens 116, 118 for allowing passage of instruments such as a fiberoptic endoscope or surgical implements, and/or for infusion and aspiration of fluids or other materials. The diameter of the access lumens 116, 118 may vary depending upon the intended application. In a presently preferred embodiment, the diameter of the access lumens 116, 118 is approximately 0.051 inch. The access lumens 116, 118 are preferably arranged generally diametrically opposite one another along a second diameter generally perpendicular to the first diameter.

The catheter shaft 44 preferably further comprises a sleeved, unitary, continuous looped steering wire tip assembly 120, which will be described with particular reference to FIGS. 6-12. The tip assembly 120 preferably comprises a first wear resistant sleeve 122 disposed at least in part within the first steering wire lumen 112 of the end segment 84, and a second wear resistant sleeve 124 disposed at least in part within the second steering wire lumen 114 of the end segment 84. The wear resistant sleeves are preferably formed from stainless steel, other metals, ceramics or other materials having a high hardness and resistance to wear. A continuous length of steering wire 126 is provided, having a first leg 128 extending through the first steering wire lumen 112, and a second leg 130 extending through the second steering wire lumen 114. In preferred form, the steering wire 126 comprises a 0.010 diameter wire. A looped segment 132 of the length of steering wire 126 extends between the first leg 128 and the second leg 130, and functions to prevent the steering wire 126 from retracting through the steering wire lumens. The first leg 128 of the steering wire 126 extends through the first wear resistant sleeve 122, and the second leg 130 extends through the second wear resistant sleeve 124. In this manner, as tension is applied to the steering wire 126 during operation, the wear-resistant sleeves 122,124 prevent the wire 126 from cutting or otherwise damaging the softer plastic material of the catheter shaft 44. In addition, the relatively harder material of construction of the end segment 84 provides improved holding of the sleeves 122,124, and improved resistance to damage from the steering wire 126 than would be provided by the relatively softer material of the tip segment 82.

In the embodiment of the present invention depicted in FIGS. 6-9, at least a portion of the looped segment 132 of the steering wire 126 is provided with an expanded outer dimension 134. The expanded portion 134 is larger in at least one dimension than the inside diameter of the opening through the wear-resistant sleeves 122, 124, so that the sleeves 122,124 and the expanded portion 134 of the steering wire 126 cooperate to resist steering wire pull-through. In a preferred embodiment, the expanded portion 134 of the steering wire 126 is a coined portion formed by crimping to deform the wire 126. The expanded outer dimension 134 is preferably in the direction of the plane of the steering wire 126, as seen best with reference to FIG. 8. In this manner, the thickness of the expanded portion, when viewed end-on as shown in FIG. 9, is reduced, thereby reducing or eliminating any potential interference by the steering wire 126 with the openings of the access lumens 116, 118. In a preferred embodiment, the entire looped segment 132 of the steering wire 126, between the first wear-resistant sleeve 122 and the second wear-resistant sleeve 124, is coined to have an expanded outer dimension in the plane of the steering wire 126. In an alternate embodiment shown in FIGS. 10-12, the looped segment 132 of the steering wire 126 is not provided with an expanded outer dimension. Encapsulation of the looped segment 132 of the steering wire 126 between the end segment 84 and the cover segment 86 further affixes the steering wire 126 in place, providing additional resistance to steering wire pull-through.

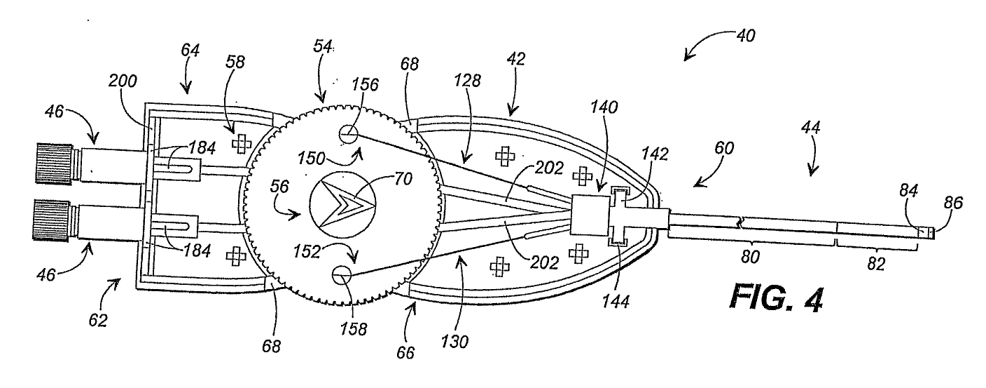

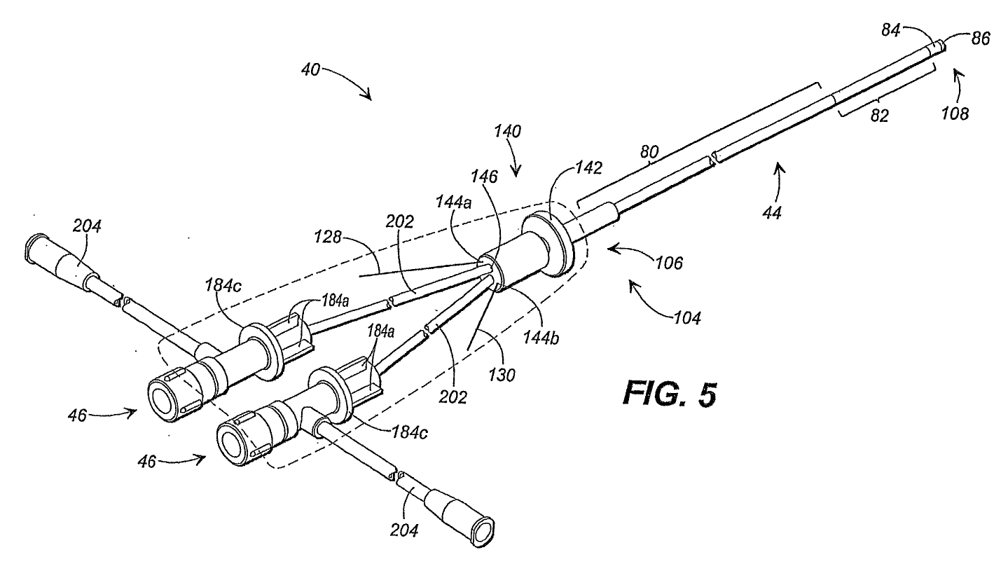

In preferred form, the catheter shaft 44 further comprises a manifold 140, as shown in FIGS. 4 and 5. The manifold 140 is coupled to the rear distal end 90 of the first shaft segment 80. Mounting means, such as a radially projecting flange 142, are preferably provided on the outer surface of the manifold 140 for attaching the catheter shaft 44 to the catheter body housing 42. For example, in the depicted embodiment, the flange 142 is adhesively secured within cooperating channel elements 144 provided within the upper and lower housing shells 50, 52 adjacent the forward end 60. The manifold 140 preferably further comprises one or more steering wire passages 144 extending therethrough, and communicating with the steering wire lumens 112,114 of the shaft assembly 104. The manifold 140 preferably further comprises one or more access passages 146 extending therethrough, and communicating with the access lumens 116,118 of the shaft assembly 104.

As seen best with reference to FIG. 4, a first free end 150 of the first leg 128 of the steering wire 126 is attached to a first connection point of the steering actuator, and a second free end 152 of the second leg 130 of the steering wire 126 is attached to a second connection point of steering actuator. For example, in the depicted embodiment, the first and second connection points of the steering actuator comprise slotted posts 156,158 projecting from the steering dial 54. The free ends 150,152 are inserted into the slots, and secured therein with hot melt or other adhesive. The slotted posts 156,158 are preferably mounted generally diametrically opposite one another on the dial 54. The steering wire 126 thus extends continuously from the first free end 150 attached to the dial 54, through a first steering wire passage 144a of the manifold 140, through the first steering wire lumen 112 of the shaft assembly 104, through the first wear-resistant sleeve 122, to the looped segment 132 adjacent the free end 108, and loops back through the second wear-resistant sleeve 124, through the second steering wire lumen 114 of the shaft assembly 104, through a second steering wire passage 144b of the manifold 140, to the second free end 152 attached to the dial 54 generally opposite the first free end 150. So arranged, rotation of the steering dial 54 about axis 56 in a first rotational direction places one leg of the steering wire in tension and the other leg in compression, thereby causing displacement of the free end 108 of the shaft assembly in a first direction. In similar fashion, rotation of the steering dial 54 about axis 56 in a second rotational direction opposite the first direction causes displacement of the free end 108 of the shaft assembly in a second direction opposite the first direction.

Alternative Catheter Tip Assemblies

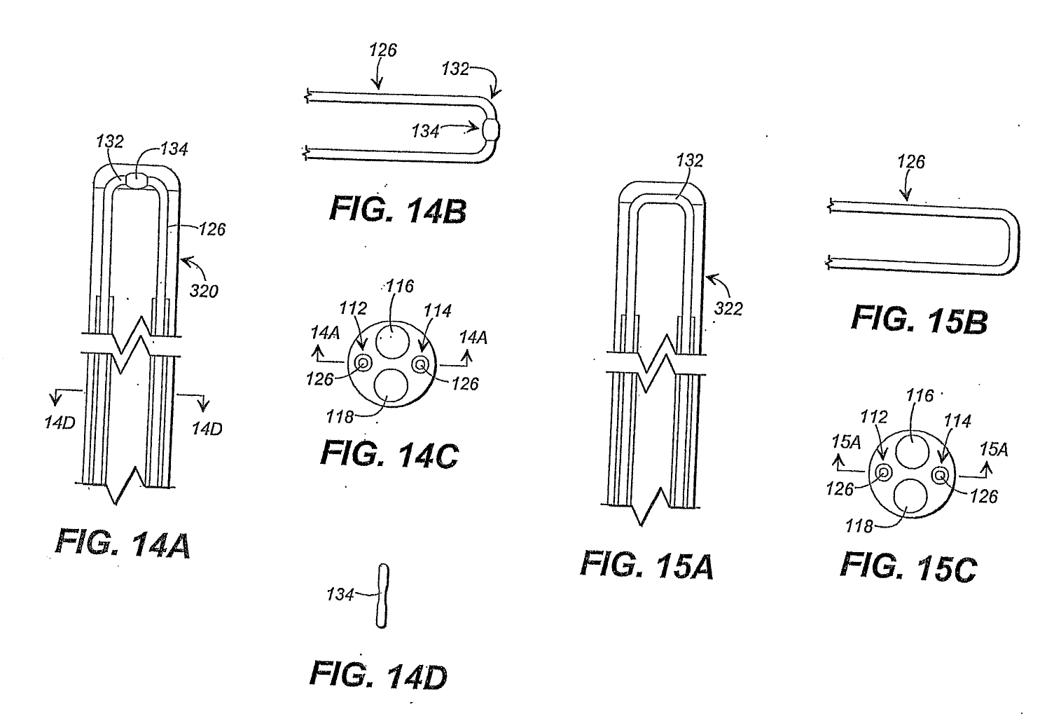

The provision of a sleeved, unitary, continuous looped steering wire tip assembly, as described above, has been found to provide superior resistance to steering wire pull-through. A number of alternative tip assemblies can, however, be substituted for the sleeved, unitary, continuous looped steering wire tip assembly, and may provide adequate performance for certain applications. For example, FIGS. 14A-D show an unsleeved looped tip assembly 320, substantially similar to the tip assembly described above with reference to FIGS. 6-9, having an expanded outer dimension 134, but without the first and second wear resistant sleeves. FIGS. 15A-C show an unsleeved looped tip assembly 322, substantially similar to the tip assembly described above with reference to FIGS. 10-12, but without the first and second wear resistant sleeves.

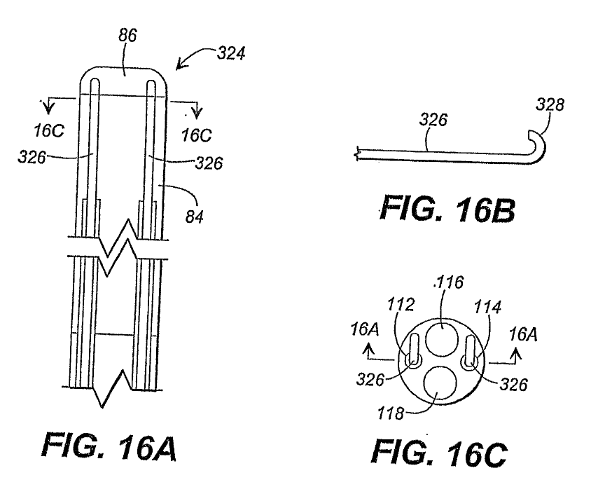

FIGS. 16A-C show a multiple hooked steering wire tip assembly 324. Two or more separate hooked steering wires 326 are provided, each with small bends formed on one end to create a hook portion 328. Each steering wire 326 passes through a steering wire lumen, and the end of each steering wire opposite the hook portion 328 is coupled to the steering actuator. The hook portion 328 abuts the second end of the end segment 84, and is dimensioned to prevent the hook portion 328 of the hooked steering wires 326 from being retracted through the steering wire lumens 112,114. The cover segment 86 encapsulates the hook portion 328 of the hooked steering wires 326.

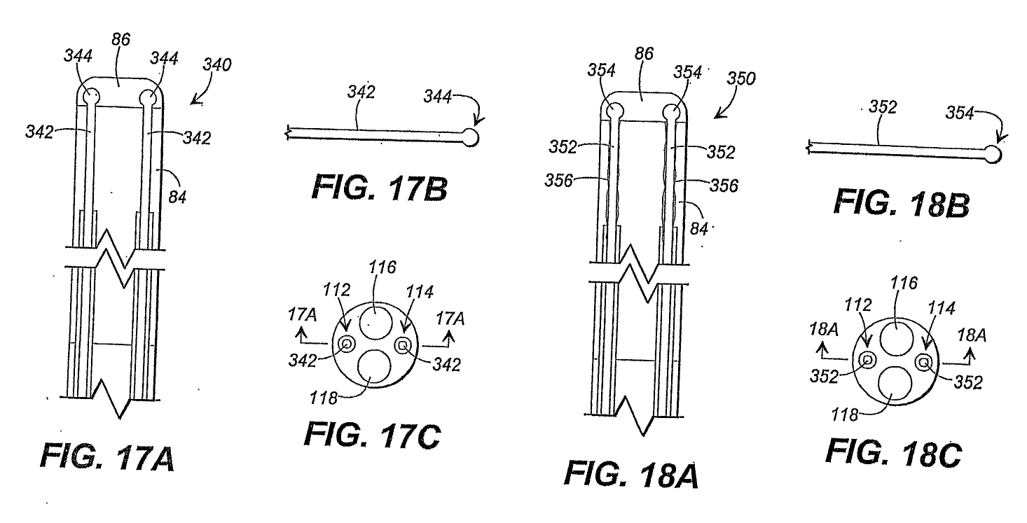

FIGS. 17A-C show a ballized wire steering wire tip assembly 340. Two or more separate ballized steering wires 342 are provided, each with a ball 344 securely affixed at a front distal end thereof. Each steering wire 342 passes through a steering wire lumen, and the end of each steering wire opposite the ball 344 is coupled to the steering actuator. The balls 344 are preferably formed of steel or other substantially rigid material, and have a diameter larger than the diameter of the steering wire lumens 112, 114, to prevent the ballized steering wires 342 from being retracted through the steering wire lumens 112, 114. In a preferred embodiment, the balls 344 are approximately 0.020″ in diameter, and the steering wire lumens 112, 114 are approximately 0.014″ in diameter. The balls 344 are preferably welded to the distal end of the ballized steering wires 342, or integrally formed therewith. The balls 344 abut the second end of the end segment 84, and are encapsulated by the cover segment 86.

FIGS. 18A-C show a jacketed ballized wire steering wire tip assembly 350. The jacketed ballized wire steering wire tip assembly 350 is substantially similar to the ballized wire steering wire tip assembly 340 described above with reference to FIGS. 17A-C, with the addition of an epoxy wire jacket 356. The jacketed ballized wire steering wire tip assembly 350 includes two or more ballized steering wires 352 extending through the steering wire lumens 112,114, each steering wire 352 including a ball 354 dimensioned to prevent the ballized steering wires 352 from being retracted through the steering wire lumens 112, 114. The epoxy wire jacket 356 is formed by injecting a curable epoxy glue into the steering wire lumens 112,114 prior to insertion of the steering wires 352. The epoxy glue is UV cured to complete the epoxy wire jacket 356. The balls 354 abut the second end of the end segment 84, and are encapsulated by the cover segment 86.

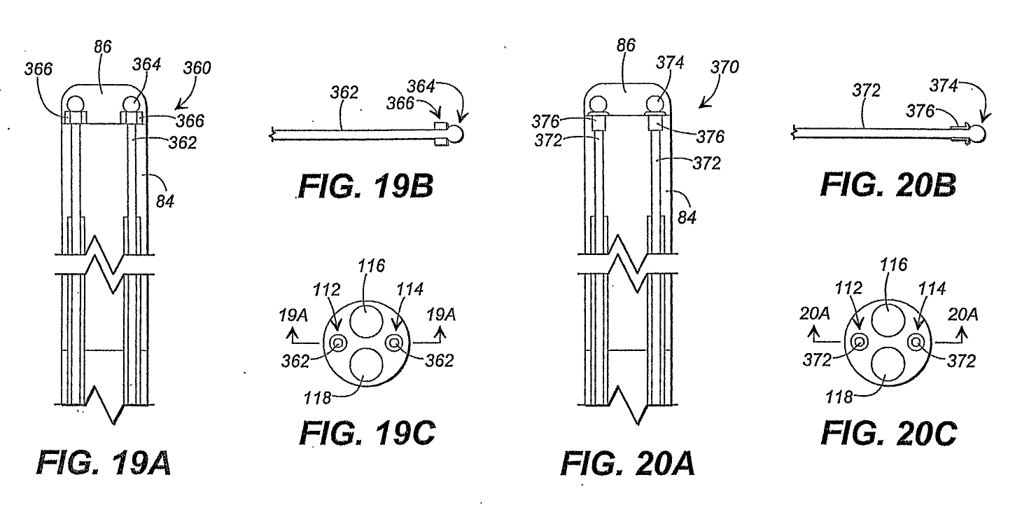

FIGS. 19A-C show a washered multiple steering wire tip assembly 360. The tip assembly 360 preferably includes ballized steering wires 362 with balls 364 affixed thereon in a manner substantially similar to the tip assembly 340 described above with reference to FIGS. 17A-C, with the addition of wire washers 366. The wire washers 366 prevent the ballized steering wires 362 from being retracted through the steering wire lumens 112, 114. A wire washer 366 is securely positioned around each ballized wire 362, and abuts the ball 364 affixed to the wire. The inside diameter of the wire washer 366 is smaller than the diameter of the ball 364, thereby preventing the ball 364 from pulling through the washer 366. The outside diameter of the washer 366 is larger than the diameter of the steering wire lumens 112, 114, thereby preventing the wire washer 366 from pulling through the steering wire lumens. In a preferred form, washers 366 having an outside diameter of 0.024″ and an inside diameter of 0.012″ are installed around a 0.010″ diameter steering wire 362 having a 0.020″ diameter ball 364 affixed thereto, for insertion into steering wire lumens 112, 114 having a 0.014 diameter. The washers 366 abut the second end of the end segment 84, and the washers 366 and balls 364 are encapsulated by the cover segment 86.

FIGS. 20A-C show an eyeleted multiple steering wire tip assembly 370. The tip assembly 370 preferably includes ballized steering wires 372 with balls 374 affixed thereon in a manner substantially similar to the tip assembly 340 described above with reference to FIGS. 17A-C, with the addition of wire eyelets 376. The wire eyelets 376 prevent the ballized steering wires 372 from being retracted through the steering wire lumens 112, 114. A wire eyelet 376 is securely positioned around each ballized wire 372, and abuts the ball 374 affixed to the wire. The inside diameter of the wire eyelet 376 is smaller than the diameter of the ball 374, thereby preventing the ball 374 from pulling through the eyelet 376. A wide portion of the eyelet 376 has an outside diameter larger than the diameter of the steering wire lumens 112, 114, thereby preventing the wide portion of the wire eyelet 376 from pulling through the steering wire lumen. A narrow portion of the eyelet 376 has an outside diameter approximately equal to or slightly larger than the diameter of the steering wire lumens 112, 114, thereby permitting the narrow portion of the wire eyelet 376 to be press fit into the steering wire lumen. In a preferred form, eyelets 376 having a wide portion with an outside diameter of 0.024″ and an inside diameter of 0.012″ are installed around a 0.010″ diameter steering wire 372 having a 0.020″ diameter ball 374 affixed thereto, for insertion into steering wire lumens 112, 114 having a 0.014 diameter. The eyelets 376 abut the second end of the end segment 84, and the eyelets 376 and balls 374 are encapsulated by the cover segment 86.

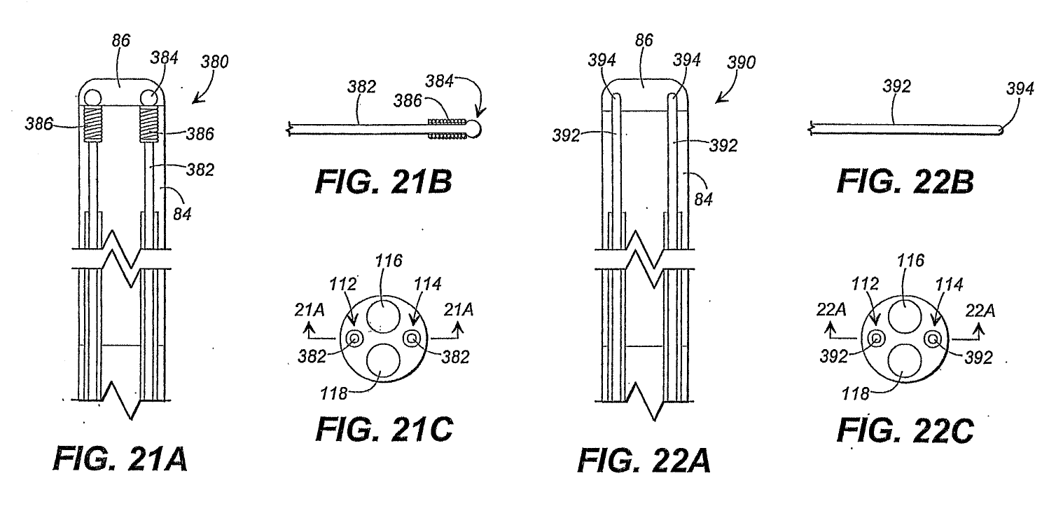

FIGS. 21A-C, show a wire springed multiple steering wire tip assembly 380. The tip assembly 380 preferably includes ballized steering wires 382 with balls 384 affixed thereon in a manner substantially similar to the tip assembly 340 described above with reference to FIGS. 17A-C, with the addition of wire springs 386. The wire springs 386 prevent the ballized steering wires 382 from being retracted through the steering wire lumens 112,114. A wire spring 386 is securely positioned around each ballized wire 382, and abuts the ball 384 affixed to the wire. The inside diameter of the wire spring 386 is smaller than the diameter of the ball 384, thereby preventing the ball 384 from pulling through the spring 386. The spring 386 has an outside diameter approximately equal to or slightly larger than the diameter of the steering wire lumens 112,114, thereby permitting the spring 386 to be press fit into the steering wire lumen. The balls 384 abut the second end of the end segment 84, and are encapsulated by the cover segment 86.

FIGS. 22A-C show a bonded steering wire tip assembly 390. Separate steering wires 392 are installed in each steering wire lumen 112,114. In a preferred form, the steering wires 392 are plastic wires approximately 0.010″ in diameter. The free ends 394 of the steering wires 392 are securely bonded, as by thermal welding or adhesives, to the end segment 84 and the cover segment 86 of the shaft, thereby preventing the steering wires 392 from retracting through the steering wire lumens. Each steering wire 392 passes through a steering wire lumen, and the end of each steering wire opposite the free end 394 is coupled to the steering actuator.

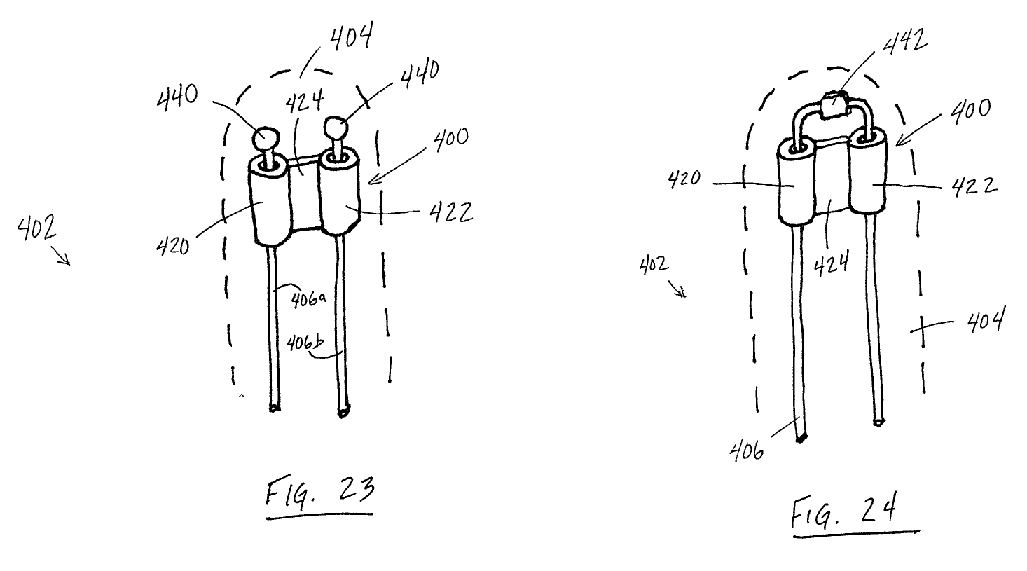

FIGS. 23-25 depict a further preferred embodiment of the present invention adapted to provide improved resistance to steering wire pull-through. The tip 410 of a catheter shaft 412 (depicted in phantom lines

Inlet Housing

The present invention preferably further provides an improved inlet housing 46. As seen best with reference to FIG. 13, the inlet housing 46 preferably comprises a unitary body portion 172 having an instrument inlet 174, an outlet 176, an internal passage 178 extending between the instrument inlet 174 and the outlet 176, and an outer mounting surface 180. The unitary body portion 172 preferably comprises a flush port 182 in fluid communication with the internal passage 178, for allowing passage of fluids such as, for example, saline fluid, pharmaceuticals, anesthetics, biologically active materials, markers, or other materials. As depicted, the flush port 182 extends outwardly from the exterior surface of the inlet housing 46, generally perpendicular to the direction of the internal passage 178, to form a generally T-shaped component. The flush port 182 can alternatively extend outwardly at an angle to form a generally Y-shaped component. A check valve, flow restricting orifice, and/or other flow control devices (unshown) can optionally be provided in the flush port 182.

The outer mounting surface 180 of the inlet housing 46 preferably comprises one or more mounting flanges 184 extending outwardly from the unitary body portion, for attaching the inlet housing 46 to an external structure or device such as, for example, the catheter body 42 described above. In the embodiment depicted in the figures, a circumferential mounting flange 184c, and a plurality of axial mounting flanges 184a, are provided. In preferred form, four axial mounting flanges 184a are provided, spaced circumferentially about the unitary body portion at approximately 900 intervals. One of the axial mounting flanges 184a is preferably generally aligned with the flush port 182, thereby enabling attachment of the inlet housing 46 to an external structure with the flush port 182 aligned generally parallel to or generally perpendicular to a mounting component of the external structure. Because the unitary body portion 172 of the inlet housing 46 of the present invention comprises a single component formed of a substantially rigid material, the flush port 182 and the mounting flanges 184 are substantially fixed in position relative to one another. In this manner, the inlet housing 46 of the present invention eliminates certain disadvantages found to result from the rotational movement permitted between the housing components of prior art Touhy-Borst fittings joined by threaded connections.

The inlet housing 46 preferably further comprises a sealing element within the internal passage, for example, adjacent the instrument inlet 174. In preferred form, the sealing element comprises a check valve such as an elastomeric duck-bill valve 186. The duck-bill valve 186 can be retained in place within the internal passage by means of a washer 188, a bushing 190, and a retaining cap 192 capable of attachment to the unitary body portion 172 such as by a threaded coupling.

The internal passage 178 of the inlet housing 46 preferably comprises a generally smooth transition throughout its entire length, from the instrument inlet 174 to the outlet 176. As used herein, “generally smooth transition” is intended to mean that the passage 178 presents no reductions in internal dimension, in the direction A of instrument insertion, at an angle greater than approximately 30° measured relative to an immediately adjacent wall surface of the internal passage.

The steerable catheter 40 of the present invention preferably comprises at least one inlet housing 46. For example, and with reference to FIGS. 2-5, two inlet housings 46 are preferably mounted within the catheter body 42 adjacent the rear end 62, with their instrument inlets 174 and flush ports 182 externally accessible. One inlet housing 46 is preferably provided for each access lumen 116,118 in the shaft assembly 104. Engaging recesses 200 are preferably provided in the upper and lower housing shells 50, 52 to receive mounting flanges 184. The cooperating flanges 184 are attached within the recesses 200, preferably by adhesive, compression fit, thermal welding or other attachment means, thereby rigidly fixing the position of the inlet housings 46 and, if provided, the flush ports 182 thereof, relative to the catheter body 42. An access conduit such as a proximal extension 202 extends between each inlet housing and a corresponding access passage 146 in the manifold 140, providing communication and passage of instruments, fluids and other objects and materials between the internal passage 178 of each inlet housing 46 and the corresponding access lumen 116,118 of the shaft assembly 104. In addition, a side extension 204 can be provided extending from each flush port 182, for connection to an external fluid source. In preferred form, the proximal extensions 202 and the side extensions 204 are preferably formed from a smooth-walled, flexible plastic tubing.

Method of Fabrication

The present invention further comprises a method of forming a segmented catheter shaft, the shaft being substantially similar to the segmented catheter shaft 44 described above. A first shaft segment, a tip segment, and an end segment are provided, each having first and second steering wire lumens extending lengthwise therethrough. The first shaft segment, the tip segment, and the end segment preferably further comprise at least one access lumen extending lengthwise therethrough. The first shaft segment is preferably formed from a material having a first stiffness. In a presently preferred embodiment, the first shaft segment is formed from a 7233 durometer PEBAX extrusion. The tip segment is preferably formed from a material having a second stiffness less than said first stiffness. In the presently preferred embodiment, the tip segment is formed from a 4033 durometer PEBAX extrusion. The end segment is preferably formed from a material having a stiffness greater than that of the tip segment. In the presently preferred embodiment, the first shaft segment is formed from a 7233 durometer PEBAX extrusion.

The first shaft segment, the tip segment, and the end segment are then bonded end-to-end, with the tip segment arranged between the first shaft segment and the end segment, to form a shaft assembly having a connecting end comprising a portion of the first shaft segment and a free end comprising a portion of the end segment. In order to prevent the internal lumens of the shaft segments from collapsing during the bonding process, mandrels are preferably inserted through the first and second steering wire lumens, and if present the access lumens, of the first shaft segment, the tip segment, and the end segment. The mandrels can comprise, for example, stainless steel rods and/or wires approximately matching the internal diameters of the lumens. According to the preferred method, the several segments of the shaft assembly are then bonded by thermal welding. The mandrels are then removed.

Optionally, a manifold can then be formed at the connecting end of the shaft assembly. The manifold is preferably formed by inserting core pins into the steering wire lumens and the access lumens at the connecting end of the shaft assembly, and injection molding plastic into a mold around the core pins to form the manifold. The core pins are then removed upon formation of the manifold. The openings remaining in the manifold after removal of the core pins form the steering wire passages and access passages through the manifold.

A continuous length of steering wire is then inserted through the steering wire lumens. Optionally, a first wear-resistant sleeve can be installed within the first steering wire lumen of the end segment, and a second wear-resistant sleeve installed within the second steering wire lumen of the end segment prior to insertion of the steering wire into the steering wire lumens. A first leg of the steering wire is inserted to extend through the first steering wire lumen, and a second leg of the steering wire is inserted to extend through the second steering wire lumen. A looped segment connects the first and second legs adjacent the tip end of the shaft assembly. The method of the present invention can optionally further comprise providing a portion of the looped segment of the steering wire with an expanded outer dimension larger than an inner dimension of the steering wire lumens, for example, by coining.

According to the preferred method, the looped segment of the steering wire is preferably encapsulated within a cover segment overlying at least a portion of the tip end of the shaft assembly. Mandrels or pins are inserted into the tip ends of the access lumens to prevent the formation of obstructions therein during the encapsulation process. A short cover segment of plastic, such as a 4033 or 7233 durometer PEBAX extrusion is mounted over the pins or mandrels, and thermal welded to the tip end of the shaft assembly. The thermal welding process encapsulates the looped segment of steering wire between the end segment and the cover segment. The pins or mandrels are then removed.

Bridged Steering Wire Sleeve Assembly

A further improvement to the present invention, described with particular reference to FIGS. 23-26, comprises a bridged steering wire sleeve element 400 for a steerable catheter 402. The catheter is substantially as described above, with a flexible catheter shaft 404 having one or more steering wire(s) 406 extending through steering wire lumens 408a, 408b thereof, and having proximal ends of the steering wire(s) attached to a steering dial in the catheter handle (unshown). One or more instrument lumens 410 are preferably provided, extending generally lengthwise through the shaft, parallel to the steering wire lumens.

The bridged steering wire sleeve element 400 is preferably mounted adjacent the tip of the catheter shaft; for example, between the end segment 84 and the cover segment 86. Alternatively, if the end segment 84 is omitted, the bridged steering wire sleeve element 400 can be installed between the tip segment 82 and the cover segment 86. In an alternate single durometer embodiment, the bridged steering wire sleeve element 400 can be mounted between the main shaft segment 80 and a cover segment 86. A standard profile extrusion segment on either or both ends of the bridged steering wire sleeve element 400 can be modified to include a slot extending between the steering wire lumens 408a, 408b to accommodate the bridged steering wire sleeve element 400 therein.

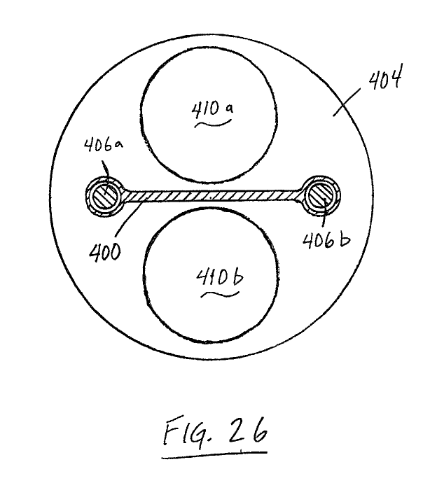

The bridged steering wire sleeve element 400 generally comprises a first sleeve portion 420, a second sleeve portion 422, and a bridge portion 424. The first sleeve portion 420 comprises a generally cylindrical hollow tube defining a first passage 430 extending coaxially and lengthwise therethrough. The second sleeve portion 422 also comprises a generally cylindrical hollow tube, generally parallel to and spaced a distance from the first sleeve portion 420, and defining a second passage 432 extending coaxially and lengthwise therethrough. The center-to-center distance between the first and second passages 430, 432 is approximately equal to the center-to-center distance between the steering wire lumens 408a, 408b. The outer diameters of the first and second sleeve portions 420, 422 are approximately equal to the inner diameters of the first and second steering wire lumens 408a, 408b. Alternatively, it may be desirable to provide first and second sleeve portions 420, 422 with slightly greater outer dimension than the inner dimension of the steering wire lumens 408a, 408b, in order to provide an interference or press fit to assist in retaining the bridged steering wire sleeve element 400 in place in the catheter shaft; or with slightly smaller outer dimension than the inner dimension of the steering wire lumens 408a, 408b, in order to provide easier assembly. In this manner, the first sleeve portion 420 is adapted to be received within the first steering wire lumen 408a and the second sleeve portion 422 is adapted to be received within the second steering wire lumen 408b without substantial deformation of the catheter shaft. The bridge portion 424 is a generally planar element joining the first and second sleeve portions 420, 422. A single bridge portion 424 can extend between the first and second sleeve portions 420, 422, along substantially the entire length of the bridged steering wire sleeve element 400, or one or more bridge portions 424 can extend along a portion of the length of the bridged steering wire sleeve element 400. For example, it may be desirable to provide a short bridging segment 424 spanning between the first and second sleeve portions 420, 422, approximately midway along the length of the bridged steering wire sleeve element 400, forming a generally “H-shaped” element. The bridge portion 424 is preferably relatively thin (e.g., 0.005″), in order to minimize the spacing required between the instrument lumens 410, and thereby maximize the instrument lumen size capable of accommodation within a catheter shaft of fixed outer dimension (see FIG. 26). In preferred form, the bridged steering wire sleeve element 400 has a length at least equal to or slightly greater than its width. The bridged steering wire sleeve element 400 is preferably fabricated as a unitary component, and is preferably formed of polycarbonate, stainless steel, or some other relatively rigid material.

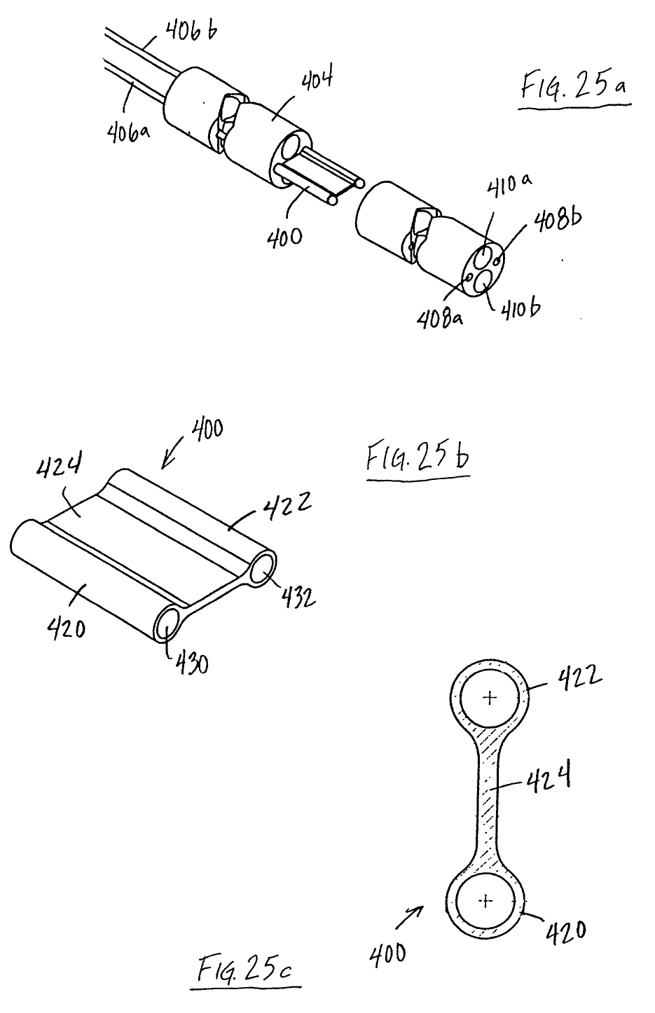

The inner diameters of the first and second passages 430, 432 are preferably slightly larger than the diameter of the steering wire(s) 406, thereby permitting the steering wire(s) to pass freely therethrough. As seen with reference to FIG. 23, in a first embodiment of the invention, distal ends of first and second steering wires 406a, 406b are provided with balls 440 or other elements or segments having a diameter larger than the inner diameter of the first and second passages 430, 432, to prevent the first and second steering wires 406a, 406b from being pulled through the passages during use. In an alternate embodiment, described with reference to FIG. 24, a single looped steering wire 406 has a coined portion 442 disposed between the first and second passages 430, 432. The coined portion has a first dimension larger than the inner diameters of the first and second passages 430, 432 to prevent the steering wire 406 from being pulled through the passages during use, and a second dimension smaller than and normal to the first dimension. The coined portion is preferably generally aligned with its first dimension in the plane of the steering wire(s), and the smaller second dimension generally perpendicular thereto, whereby the looped portion of the steering wire presents a minimal profile between the instrument lumens and thereby helps maximize the instrument lumen size capable of accommodation within a catheter shaft of fixed outer dimension. Other forms of expanded dimension segment(s) of the steering wire(s) 406 can be provided, in addition to the ballized and coined embodiments described, in alternate embodiments of the invention.

Upon assembly of the catheter with the bridged steering wire sleeve element 400 engaged within the steering wire lumens 408 of the tip of the catheter shaft 404, the bridged steering wire sleeve element 400 and the expanded dimension segment(s) of the steering wire(s) 406 cooperate to provide better steering performance and reduce the likelihood of steering wire pull-through. If the bridged steering wire sleeve element 400 is formed of polycarbonate or other thermoplastic material, the bridged steering wire sleeve element 400 may deform slightly or fuse with surrounding shaft material during fusion of the catheter tip segments, thereby providing a more secure attachment of the bridged steering wire sleeve element 400 within the catheter shaft. Alternatively or additionally, the shaft material may deform slightly around the bridged steering wire sleeve element 400 during fusion of the catheter tip segments, also providing more secure attachment.

While the invention has been described in its preferred forms, it will be readily apparent to those of ordinary skill in the art that many additions, modifications and deletions can be made thereto without departing from the spirit and scope of the invention.

What is claimed is:

1. A catheter tip assembly comprising:
at least one steering wire;
a bridged steering wire sleeve element comprising a first sleeve having a first passage extending therethrough for receiving a first steering wire portion, a second sleeve having a second passage extending therethrough for receiving a second steering wire portion, and a bridge portion extending between said first and second sleeves.

2. The catheter tip assembly of claim 1, wherein said first and second sleeves comprise generally tubular elements.

3. The catheter tip assembly of claim 1, wherein said steering wire sleeve element is formed from a material selected from the group of polycarbonate and stainless steel.

4. A catheter comprising:
a flexible shaft comprising first and second steering wire lumens;
at least one steering wire extending through said first and second steering wire lumens; and
a bridged steering wire sleeve element comprising a first sleeve engaged within said first steering wire lumen and having a first passage extending therethrough for receiving a first steering wire portion, a second sleeve engaged within said second steering wire lumen and having a second passage extending therethrough for receiving a second steering wire portion, and a bridge portion extending between said first and second sleeves.

5. The catheter of claim 4, wherein said steering wire sleeve element is formed from a plastic material adapted to bond with said flexible shaft.

6. The catheter of claim 4, wherein said at least one steering wire comprises a pair of ballized steering wires having balled ends with an outer dimension larger than an inner dimension of the first and second passages.

7. The catheter of claim 4, wherein said at least one steering wire comprises a looped steering wire having first and second legs and a looped portion therebetween.

8. The catheter of claim 7, wherein said looped portion is coined to have an outer dimension larger than an inner dimension of the first and second passages.

9. A bridged steering wire sleeve element comprising a first sleeve having a first passage extending therethrough for receiving a first steering wire portion, a second sleeve having a second passage extending therethrough for receiving a second steering wire portion, and a bridge portion extending between said first and second sleeves.

10. The steering wire sleeve element of claim 9, wherein said first and second sleeves comprise generally tubular elements.

11. The steering wire sleeve element of claim 9, wherein said steering wire sleeve element is formed from a material selected from the group of polycarbonate and stainless steel.

12. A method of resisting steering wire pull-through in a shaft of a steerable catheter, comprising providing a first sleeve engaged within a first steering wire lumen of the shaft and having a first passage extending therethrough receiving a first steering wire portion, providing a second sleeve engaged within a second steering wire lumen of the shaft and having a second passage extending therethrough receiving a second steering wire portion, and providing a bridge portion extending between said first and second sleeves.

13. A method of fabricating a catheter shaft, comprising:
providing a flexible shaft comprising first and second steering wire lumens;
installing a bridged steering wire sleeve element comprising a first sleeve engaged within said first steering wire lumen and having a first passage extending therethrough, a second sleeve engaged within said second steering wire lumen and having a second passage extending therethrough, and a bridge portion extending between said first and second sleeves; and
installing at least one steering wire into said first and second steering wire lumens and through the first and second passages of the steering wire sleeve element.
